(19) United States
(12) Patent Application Publication
(10) Pub. No.: US 2009/0012419 A1
(43) Pub. Date: Jan. 8, 2009

(54) SYSTEM AND METHOD FOR PERFORMING PHYSIOLOGICAL ASSESSMENTS

(76) Inventor: Geoffrey R. McKee, Columbia, SC (US)

Correspondence Address: MCNAIR LAW FIRM, P.A., P.O. BOX 10827, GREENVILLE, SC, 29603-0827, US

(21) Appl. No.: 12/146,843

(22) Filed: Jun. 26, 2008

Related U.S. Application Data
(60) Provisional application No. 60/958,501, filed on Jul. 6, 2007.

(51) Int. Cl. A61B 13/00; A61B 5/103

(52) U.S. Cl. 600/558; 600/595

(57) ABSTRACT

A system and method for assessing sexual attention of a test subject comprising: a computer readable medium; a display; an eye-tracking sensor; a set of display sets where each display set has images representing individuals having a plurality of different genders and age groups wherein each display set contains images having a unique combination of gender and age group; and; a set of computer readable instructions embodied in said computer readable medium for display said display sets on said display, determining the test subject's eye fixation time for each image, determining the test subjects saccade time for each displayed image, determining a gaze score according to said fixation time and said saccade time, and determining a gaze score for each combination of gender and age group.

### CLAIM OF PRIORITY

This patent application claims priority on U.S. Patent Provisional Application Ser. No. 60/958,501 entitled System and Method for Performing Physiological Assessments filed Jul. 6, 2007.

### FIELD OF THE INVENTION

This invention is directed to a system and method for performing and recording results from a physiological assessment and more specifically to a physiological assessment of sexual attention for aiding in the evaluation, diagnosis or treatment of paraphilias, sexual dysfunctions, gender identity disorders, related sexual disorders.

### BACKGROUND OF THE INVENTION

Forensic examiners are often used to provide expert opinions as to whether a test subject has a mental disorder such as a pedophilia or another paraphilia. Several states have laws which provide for the involuntary commitment and treatment of sexually violent predators (SVPs). One method of evaluating test subjects is to use the classification system described in the Diagnostic and Statistical Manual for Mental Disorders, Fourth Edition (DSM-IV). However, there is criticism to using the information from the DSM-IV since there is believed to be insufficient data concerning its reliability.

Prior to subjecting an individual to involuntary mental health treatment, it would be advantageous to ensure that the individual is truly a sexually violent predator. Generally, states have adopted the standard that prior to subjecting someone to involuntary mental health treatment, there should be a legal finding that the person has a mental abnormality or personality disorder that makes the person likely to engage in acts of sexual violence. In order to make this determination, evaluators customarily use the diagnostic categories described in the DSM-IV and the sections concerning personality disorders and paraphilia disorders in particular.

Whether or not an individual meets criteria for civil commitment is a legal finding ultimately made by a judge or jury, but the diagnostic conclusions drawn by mental health professionals are central to this process. Therefore, it is important to have a reliable method for making such diagnostic conclusions.

Research has shown that the overall recommendation decisions made by evaluators using DSM-IV standards were characterized as having “poor” reliability. These studies raised concerns about the credibility of civil commitment evaluations based upon DSM-IV and other such means. The consequences to the individual being evaluated and to public safety if incorrect determinations are made could be devastating.

Prior attempts to provide a diagnosis tool have several shortcomings. For example, the Abel Assessment of Sexual Interest-2 (AASI-2) attempts to measure sexual interest through measuring the time that a test subject views a single photograph. Photographs are displayed to the test subject in linear fashion and the time that the test subject views the photograph is recorded. However, AASI-2 is limited to this single metric. Further, AASI-2 can more easily be “fooled” since the test subject need only vary the amount of time each photograph is viewed. Results from the AASI-2 test must be sent to Abel Screening Inc before the test results can be known for the test subject thereby creating significant delay between the times where the test is given and when the test results are reported. Further, AASI-2 displayed models only a limited number of times, two in its current version, so that there is a limited ability to verify responses from test subjects.

It would be advantageous to have a system and method for testing with physiological assessment which contains multiple measures as well as measures of non-conscious reflective actions, shortened test results scoring times, and external validation of test subject responses.

### SUMMARY OF THE INVENTION

The above objectives are accomplished by providing a system and method for assessing sexual attention of a test subject comprising: a computer readable medium; a display in electronic communications with the computer readable medium; an eye-tracking sensor in electronic communications with the computer readable medium for tracking eye movement of the test subject viewing the display; a set of display sets embodied in the computer readable medium where each display set has images representing individuals having a plurality of different genders and age groups wherein each display set contains images having a unique combination of gender and age group; and; a set of computer readable instructions embodied in the computer readable medium for display the display sets on the display, determining the test subject's eye fixation time for each image, determining the test subjects saccade time for each displayed image, determining a gaze score according to the fixation time and the saccade time, and determining a gaze score for each combination of gender and age group.

The invention can include a set of base line gaze scores embodied in the computer readable medium and derived from normalized group testing; and, the set of computer readable instructions include instructions for comparing the determined gaze scores with the set of baseline gaze scores so that a comparison of the test subject's gaze scores can be compared with normalized group test results. The display set can includes an image of an adult male, an adult female, teen male, teen female, prepubertal male, prepubertal female, preschool male and preschool female. The computer readable instructions can include instructions for determining the gaze score representing how long images of an adult male, an adult female, teen male, teen female, prepubertal male, prepubertal female, preschool male and preschool female each held the test subject's gaze. The set of display sets contain images of individuals having a plurality of different ethnic origins and wearing different attire.

### DESCRIPTION OF THE DRAWINGS

The invention will be described herein with reference to the following drawings:

FIG. 1 is a diagram of components of the invention;

FIG. 2 is a representation of displayed images of the invention;

FIG. 3 is a flow chart of the invention;

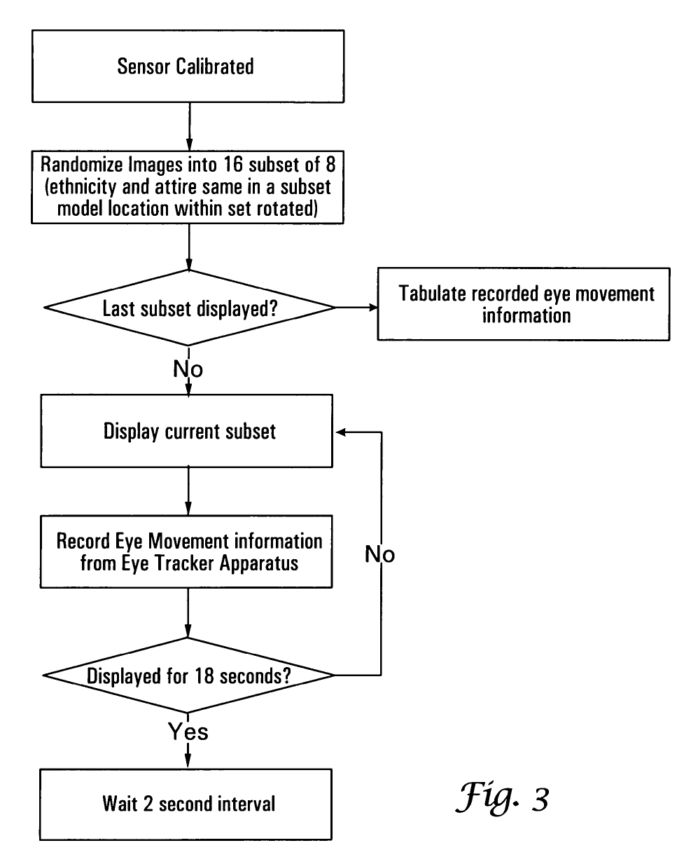

FIG. 4 is a representation of the output from the invention; and,

FIG. 5 is a representation of the output from the invention.

### DETAILED DESCRIPTION OF THE INVENTION

An object or module is a section of computer readable code embodied in a computer. The detailed description that follows may be presented in terms of program procedures executed on a computer or network of computers. These procedural descriptions are representations used by those skilled in the art to most effectively convey the substance of their work to others skilled in the art. These procedures herein described are generally a self-consistent sequence of steps leading to a desired result. These steps require physical manipulations of physical quantities such as electrical or magnetic signals capable of being stored, transferred, combined, compared, or otherwise manipulated readable medium that is designed to perform a specific task or tasks. Actual computer or executable code or computer readable code may not be contained within one file or one storage medium but may span several computers or storage mediums. The term “host” and “server” may be hardware, software, or combination of hardware and software that provides the functionality described herein.

The present invention is described below with reference to flowchart illustrations of methods, apparatus (“systems”) and computer program products according to the invention. It will be understood that each block of a flowchart illustration can be implemented by a set of computer readable instructions or code. These computer readable instructions may be loaded onto a general purpose computer, special purpose computer, or other programmable data processing apparatus to produce a machine such that the instructions will execute on a computer or other data processing apparatus to create a means for implementing the functions specified in the flowchart block or blocks.

These computer readable instructions may also be stored in a computer readable medium that can direct a computer or other programmable data processing apparatus to function in a particular manner, such that the instructions stored in a computer readable medium produce an article of manufacture including instruction means that implement the functions specified in the flowchart block or blocks. Computer program instructions may also be loaded onto a computer or other programmable apparatus to produce a computer executed process such that the instructions are executed on the computer or other programmable apparatus provide steps for implementing the functions specified in the flowchart block or blocks. Accordingly, elements of the flowchart support combinations of means for performing the special functions, combination of steps for performing the specified functions and program instruction means for performing the specified functions. It will be understood that each block of the flowchart illustrations can be implemented by special purpose hardware based computer systems that perform the specified functions, or steps, or combinations of special purpose hardware or computer instructions. The present invention is now described more fully herein with reference to the drawings in which the preferred embodiment of the invention is shown. This invention may, however, be embodied any many different forms and should not be construed as limited to the embodiment set forth herein. Rather, these embodiments are provided so that this disclosure will be thorough and complete and will fully convey the scope of the invention to those skilled in the art.

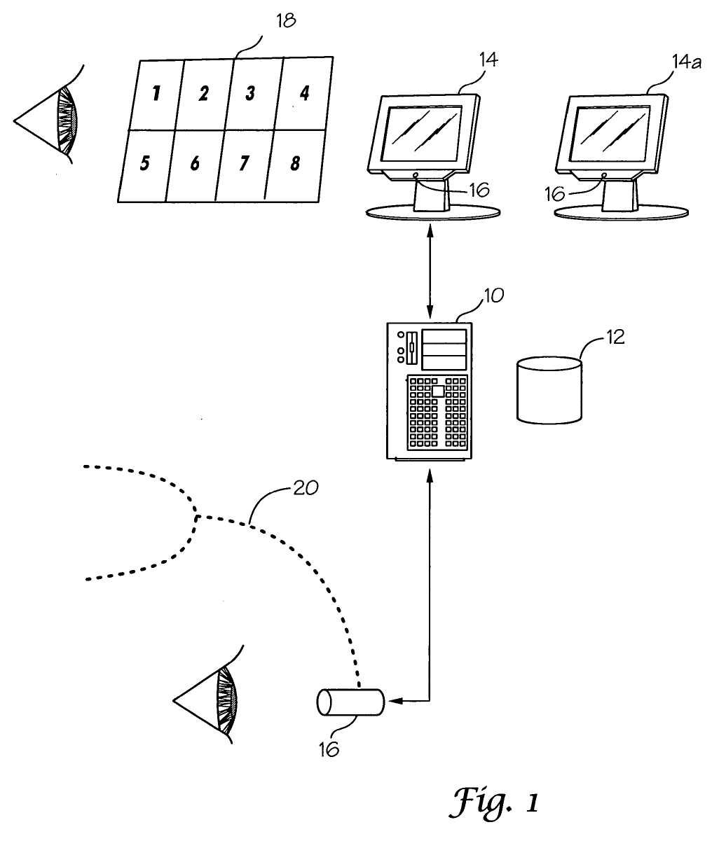

Referring to FIG. 1, a computer 10 having a computer readable medium 12 contains computer readable instructions. A display 14 is in communication with computer 10 for displaying images to a user. Referring to display 14a, a sensor 16 is able to determine an individual's pupil in order to determine the direction of the individual's gaze. Therefore, an eye tracking sensor or sensor 16 will allow computer readable instructions to determine which area of the display the user is gazing. Further, sensor 16 will also be able to determine fixation and saccade. Fixation is the amount of time that an individual gazes at a specific location. Saccade is the amount of time that transpires when a user looks from one location to another on the display.

In one embodiment, the user has eight images which are displayed on display 14. The set of images 18 can be numbered one through eight for identification purposes. The test subject can then be asked a question such as “Which of these do you find most sexually attractive?”, and can use the mouse or other input device to indicate which of the images is selected. During the time in which the user is viewing the set of images, sensor 16 is determining fixation and saccade of the test subject for each of the images. This information is transmitted to computer 10 and stored in computer readable medium 12. With fixation and saccade known for a particular image, a gaze time can be calculated for each of the displayed images.

Saccades are quick, simultaneous movements of both eyes in the same direction. Using the fixation on an image and the saccade across displayed images, a gaze time for each of the displayed images can be determined. Knowing the total display time of a display set having displayed images, a percentage of total gaze score for each image displayed can be determined.

While there are multiple methods for calculating the gaze percentage of a test subject for displayed images, one example is if it is determined that the display set is displayed for 18 seconds and one image holds the gaze of the test subject for 4 seconds, the image is calculated to have held the test subject's gaze for approximately 22% of the time ( 4/18). In this example, the gaze percentage would be 22%.

In another example, the number of fixations to a specific image can be included in the calculation. If the test subject returns his gaze to a particular image 10 times, then the number of fixations score would be 10 and this value can be used in combination with the gaze percentage score to provide more reliable data about the test subjects gaze returning to a particular image.

In one embodiment, sensor 16 is located on a desk below display 14. Sensor 16 then determines gaze of the user and saccade of the user and will transmit this information at computer 10. In one embodiment, the eye-tracking sensors are the TM3 offered by Eye Tech Digital Systems. In one embodiment, the sensor can be worn by the test subject.

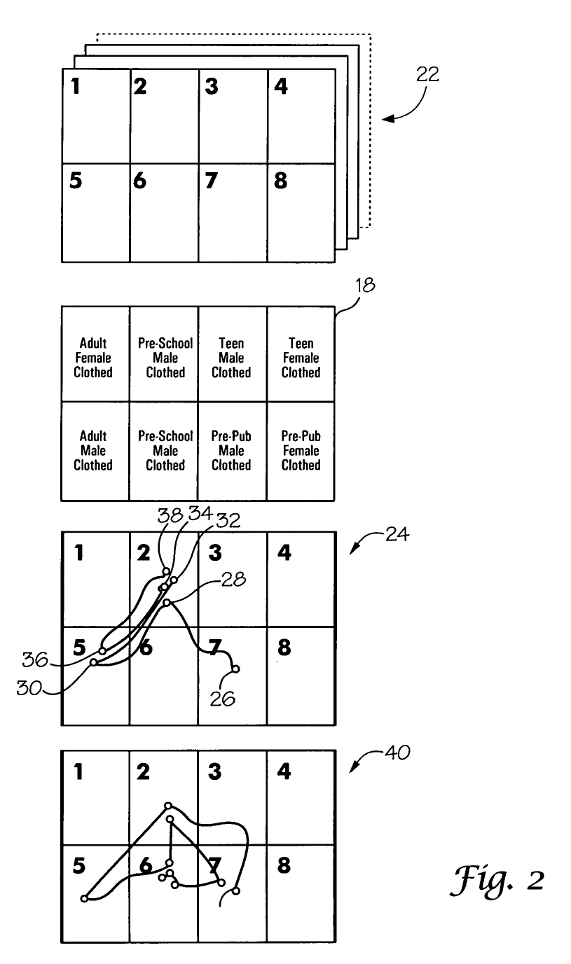

Referring now to FIG. 2, a set of images is shown generally as 22. In one embodiment, sixteen sets are used. Four different models of each gender (male and female) are used in a set. Further, the attire is held consistent across a display set and ranges from fully clothed to wearing swimsuits or undergarments. Each set also contains four age groups which can be described as adult, teenage, prepubertal, and preschool. Generally, prepubertal is the age range between eight to thirteen, while preschool is under eight years old. Each set can also be of a different ethnic origin and the ethnic origins can be held constant across a particular display set. Ethnic origins can include Caucasian, African-American, Hispanic, or other ethnic origins. Therefore, each set would contain four males and four females, two adults, two teenagers, two prepubertal and two preschool individuals and have ethnicity and attire consistent. In one embodiment, two ethnicities are used and two attires are used which result in sixteen sets of eight or 128 images.

Advantages that are offered by this invention are that images are presented in groups rather than one at a time and the test subject's fixation duration, number of fixations to each image and saccades between each image can be measured. It does not rely on simply measuring how long, or fixation, a test subject has with a single image. Although fixation can be measured for an image of the group, the invention also allows the measurement of saccade between the images.

For example, results from the sensor 16 are shown generally as 24 of FIG. 2. It can be shown the test subject initially gazes upon image 7 having an initial point 26. The amount of time the individual gazes at image 7 is recorded in the computer readable medium. The test subject then moves the gaze to point 28 which allows the invention to measure both the amount of time it took for the test subject to move to point 28 as well as how long the test subject gazed at point 28. The test subject then moves to point 30, 32, 34, 36, and 38, respectively, with the amount of time measured when the gaze shifts as well as the fixation on the particular images. As can be seen, this test subject had a fixed gaze, mostly at image 2. If image 2 represents an adult female and the test subject is male, the results would be typical for a heterosexual male's sexual attention. However, if image 2 was a preschool male, it would indicate there may be an atypical sexual attention propensity to boys in this test subject. Test results shown as 40 show that the saccade of the test subject follows a greater path, but again shows that the test subject gazed upon image 6 more than the remainder of the images. Further, it can be shown that the saccade of the test subject caused a viewing path to travel through image 3, but the test subject did not fix his gaze on image 3 for any appreciable period of time. Based upon the fixation durations, number of fixations and saccade durations measured by the invention and relationship of these measures with the particular type of image displayed, an indication can be given toward the sexual attention of the test subject.

In addition to the fixation duration determined by sensor 16, the invention is also able to determine the number of times the test subject visually fixates on a particular image. Further, since multiple sets of images are provided, the time the test subject views each model or type of model can be determined. The sets of images also allow for the progression of the test subject's gaze so that the sequence of images can be determined as viewed by the test subject. This can be helpful in showing if a test subject re-fixates on an image previously viewed since sets of images are displayed.

It should also be noted that in one embodiment and according to research and literature on sexual interests, ethnicity is consistent across each set since there is some indication that adult males tend to have sexual interests and attention in persons of their same ethnicity by making the test having the ability to be tailored toward the ethnicity of the test subject.

Further advantages provided by this invention are that measurements are done in near real-time and therefore can calculate unconscious, reflexive eye movements beyond a test subject's awareness. This can provide for more accurate and valid indications of sexual interest since fixation and saccade as well as image progression and other recorded information are much more difficult to consciously control. This invention also has the ability to generate group information allowing normalized test ranges which can help establishing base lines for using this invention.

Further, the test results of this invention can be immediately knowable to the clinician or individual administering the tests without the need to transmit the measurements to any third party and await an interpretation or diagnosis. Further, the results can be independently verified by other clinicians or researchers since the scoring of the invention can be repeated.

This invention can assist in psychological testing to aid in the evaluation, diagnosis, and treatment of pedophilia in both adult and adolescent males and although much rarer, in adult and adolescent females. This invention may also help to empirically validate the diagnosis of a person's particular pedophilia as well as a test subject's type of pedophilia. This invention can also be used for adolescents and can be helpful in the early diagnosis of pedophilia allowing a clinician much longer time for treatment which may reduce the likelihood of adult sexual recidivism, particularly against children.

This invention also has applications as a screening tool to determine the pedophilic interests of job applicants. Clearly, there are some jobs such as daycare, child protective services, juvenile justice, handicap services, education field, and other industries in which knowing whether an individual has unacceptable pedophilic interests would be of great value.

In use of the invention, the sensor is calibrated at 42. The images are then randomized into sixteen subsets with ethnicity and attire remaining constant and having four male, four female, in two of each age group, in each of the sixteen subsets at 44. If the last subset is displayed at 46, the information recorded from the test subject viewing the images is made at 48. If the last subset of images is not displayed, the current subset is displayed at 50. The eye movement information including fixation and saccade is measured at 52. If the set has been displayed for eighteen seconds at 54, at a two-second interval, then the invention moves to the next subset at 56. The process returns to 46 to determine if that was the last subset displayed. If the subset has not been displayed for eighteen seconds, it remains viewable by the test subject and eye movement information continues to be recorded at 52.

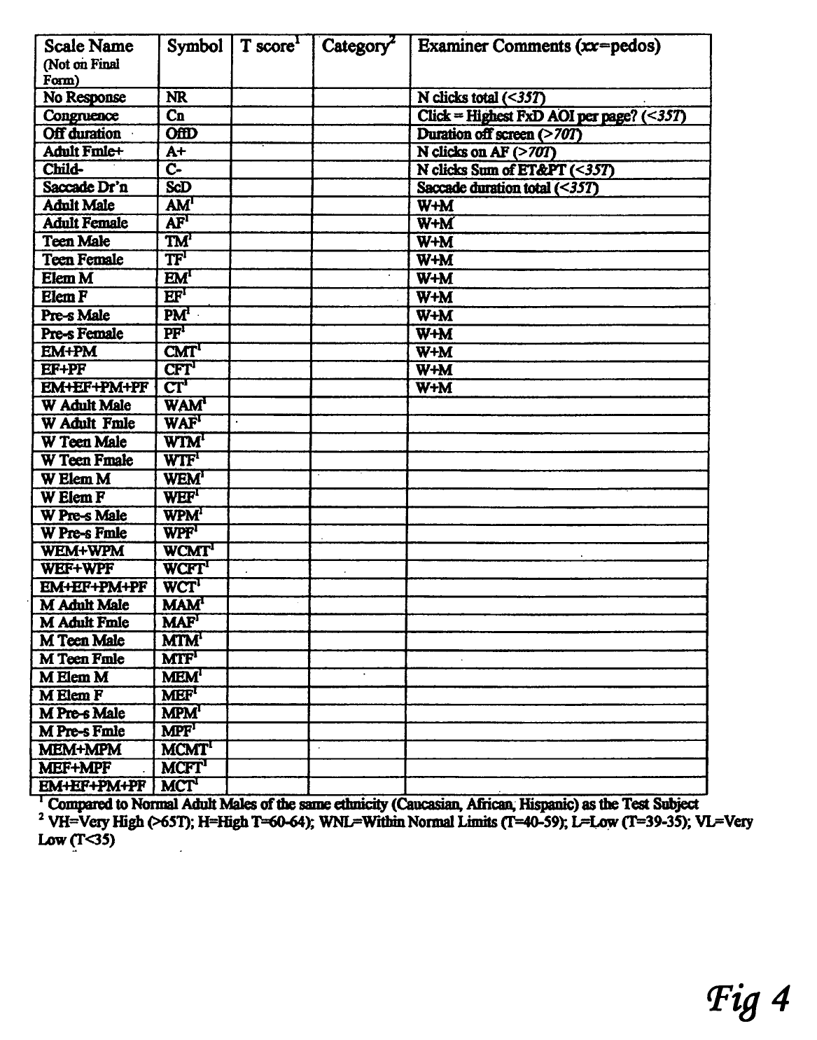

Referring to FIG. 4, an example of test results is given. There are several scales which can be used. “NR” represents no response; “Cn” represents congruence; “OffD” represents off duration; “A+” represents Adult female+; “C−” represents child−; “ScD” represents saccade duration. There are also a number of clinical scales which can measure the subject's eye movement and can include fixation duration (FxD) or number of fixations (NFx) to each of the various model types and images viewed. The invention can also provide composite clinical scales that focus on same sex children of certain age groups (CMT) (CFT) as well as boys and girls in a single age group (PM+PF) and non-adolescent boys and girls (CT). The test subjects can be compared to normals of the same ethnicity so as to remove any data distortion between ethnicities. These scores can then be reported which would represent the test subject's results per the scale compared to the average test score of a normal group. This will allow the evaluator or clinician to quickly interpret the text subject's sexual interest and can be used to formulate categories of sexual interest in children as being very high, high, within normal limits, low, or very low. This allows for quick and easy determinations of the results.

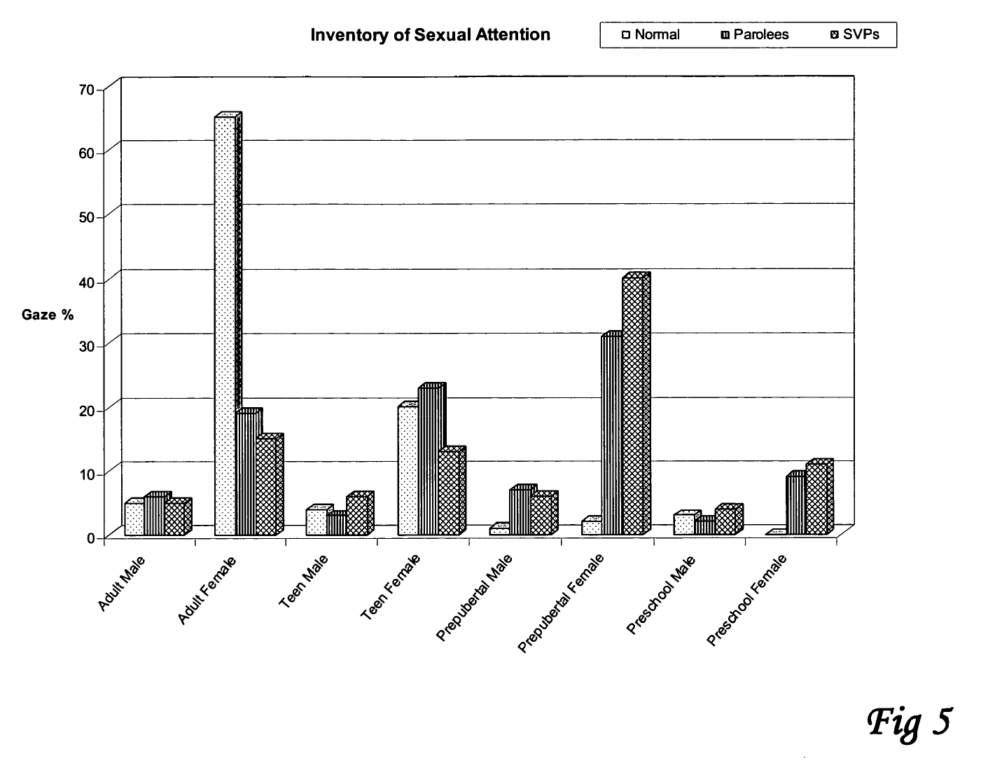

Referring to FIG. 5, an example of cumulation of results is shown. As can be shown, for a normal male, over sixty percent of the gaze was spent on adult females from results taken from this invention. However, for sexually violent predators (SVP), almost forty percent of the gaze percentage was spent on prepubertal females.

This invention can also use a Tobii E-17 eye tracking device in order to determine the gaze and saccade of a test subject.

This invention can record initial fixation which is the first duration and location of the test subject's gaze. The initial fixation to one of the images of the set of images is the first fixation, then saccade to another image of the set for the second fixation which is the amount of time the test subject gazes at the second image, then a saccade back to the first image, or another image, which would be third fixation, and so on. The frequency, consistency, fixation, duration, and pattern of re-fixation of the test subject's gaze provides information to indicate the individual's sexual attention preferences for males and/or females of varying ages.

This invention can also be used to assist and aid in the evaluation, diagnosis, and treatment of paraphilias (especially pedophiles) of certain sexual dysfunctions, gender identification disorders, and related sexual disorders such as ego-dystonic gender orientation.

This invention may also be helpful to determine whether the sexual attention of an individual is changing. Since this is an objective way of measuring the psychological treatment of a test subject's pedophilia, it can be measured to determine if there are improvements based upon treatment or other factors.

This invention can enhance psychiatrists, psychologists, and other health care providers to diagnose and perform forensic analysis concerning test subjects who have been charged or convicted of sexually violent predator acts and/or have become subject to post-incarceration involuntary treatment in the various states.

While a preferred embodiment of the invention has been described using specific terms, such description is for illustrative purposes only, and it is to be understood that changes and variations may be made without departing from the spirit or scope of the following claims.

What is claimed is:

1. A system for assessing sexual attention of a test subject comprising:
a computer readable medium;
a display in electronic communications with said computer readable medium;
an eye-tracking sensor in electronic communications with said computer readable medium for tracking eye movement of the test subject viewing said display;
a set of images embodied in said computer readable medium representing individuals having a plurality of different genders and age groups; and;
a set of computer readable instructions embodied in said computer readable medium for determining a display set having a plurality of images selected from said set of images wherein each of said displayed images is a unique combination of gender and age group, displaying said display set using said display to the test subject, determining the test subject's eye movement over the displayed images on the display by determining the test subject's fixation duration for each image and determining a gaze percentage of the total display time said display set was displayed that each images held the test subject's gaze so that the percentage of said total display time for each combination of gender and age group that held the test subject's gaze is known.

2. The system of claim 1 wherein said computer readable instructions include instructions for determining a saccade duration between each displayed image representing the time the test subject's gaze moves from one said displayed image and to another said displayed images and determining a gaze score according to said saccade duration and said gaze percentage.

3. The system of claim 2 wherein said computer readable instructions include instructions for determining the number of fixations for each displayed image representing the number of times that test subject's gaze was held by each image moves and said computer readable instruction for determining a saccade score include determining a gaze score according to said saccade duration, said gaze percentage and said number of fixations.

4. The system of claim 1 including:
a set of base line gaze percentages embodied in said computer readable medium derived from normalized group testing; and,
said set of computer readable instructions include instructions for comparing said determined gaze percentages with said set of baseline gaze percentages so that a comparison of the test subject's gaze percentages can be compared with normalized group test results.

5. The system of claim 2 including:
a set of base line gaze scores embodied in said computer readable medium derived from normalized group testing; and,
said set of computer readable instructions include instructions for comparing said determined gaze scores with said set of baseline gaze scores so that a comparison of the test subject's gaze scores can be compared with normalized group test results.

6. The system of claim 1 wherein said computer readable instructions include instructions for determining a plurality of display sets, displaying to the test subject each display set using said display, determining a gaze percentage of the total display time said display sets were displayed of each image that held the test subject's gaze so that said gaze percentage of said total display time for each combination of gender and age group that held the test subject's gaze is known.

7. The system of claim 1 wherein said computer readable instructions include instructions for determining a plurality of display sets, displaying to the test subject each display set using said display, determining a saccade duration between each displayed image representing the time the test subject's gaze moves to said displayed image and determining a gaze score according to said gaze percentage and said saccade time between each image displayed.

8. The system of claim 1 wherein said display set includes an image of an adult male, an adult female, teen male, teen female, prepubertal male, prepubertal female, preschool male and preschool female.

9. The system of claim 8 where said computer readable instructions include instructions for determining the gaze percentage of total display time that images of an adult male, an adult female, teen male, teen female, prepubertal male, prepubertal female, preschool male and preschool female each held the test subject's gaze.

10. The system of claim 1 wherein:
said set of images representing individuals having a plurality of ethnic origins; and,
said set of computer readable instructions includes instructions for determining a display set having images of individuals having a plurality of ethnic origins.

11. The system of claim 10 wherein said computer readable instructions for determining a plurality of display sets include instructions for determining a plurality of display sets wherein each images in a single display set contains images of the same ethnic origin.

12. The system of claim 1 wherein:
said set of images representing individuals wearing varying attire; and,
set of computer readable instructions includes instructions for determining a display set having images of individuals wearing varying attire.

13. The system of clam 12 wherein said computer readable instructions for determining a plurality of display sets include instructions for determining a plurality of display sets wherein each images in a single display set contains images of individuals wearing the same attire.

14. The system of claim 1 wherein said display set includes eight images.

15. A system for assessing sexual attention of a test subject comprising:
a computer readable medium;
a display in electronic communications with said computer readable medium;
an eye-tracking sensor in electronic communications with said computer readable medium for tracking eye movement of the test subject viewing said display;
a set of display sets embodied in said computer readable medium where each display set has images representing individuals having a plurality of different genders and age groups wherein each display set contains images having a unique combination of gender and age group; and;
a set of computer readable instructions embodied in said computer readable medium for display said display sets on said display, determining the test subject's fixation duration ime for each image, determining the test subjects saccade duration between each displayed image, determining a gaze score for each image according to said fixation duration and said saccade duration, and determining a gaze score for each combination of gender and age group.

16. The system of claim 15 wherein said computer readable instructions include instruction for determining the number of fixations for each displayed image, and said computer readable instructions for determining a gaze score includes instructions for determining a gaze score according to said fixation duration, said saccade duration and said number of fixations.

17. The system of claim 15 including:
a set of base line gaze scores embodied in said computer readable medium and derived from normalized group testing; and,
said set of computer readable instructions include instructions for comparing said determined gaze scores with said set of baseline gaze scores so that a comparison of the test subject's gaze scores can be compared with normalized group test results.

18. The system of claim 15 wherein said display set includes an image of an adult male, an adult female, teen male, teen female, prepubertal male, prepubertal female, preschool male and preschool female.

19. The system of claim 18 where said computer readable instructions include instructions for determining the gaze score representing how long images of an adult male, an adult female, teen male, teen female, prepubertal male, prepubertal female, preschool male and preschool female each held the test subject's gaze.

20. The system of claim 15 wherein:
said set of display sets contain images of individuals having a plurality of different ethnic origins; and,
each display set contains images of individuals having a the same ethnic origins.

21. The system of claim 15 wherein:
said set of display sets contain images of individuals wearing differing attire; and,
each display set contains images of individuals wearing the same attire.

22. The system of claim 15 wherein each display set includes eight images.

23. A method for assessing sexual attention of a test subject comprising:
providing an eye-tracking sensor for tracking eye movement of the test subject;
providing a set of display sets representing individuals having a plurality of different genders and age groups wherein each display set contains images having a unique combination of gender and age group;
displaying said display sets to the test subject for a predetermined period of time;
determining the test subject's fixation duration for each image using said eye-tracking sensor;
determining a gaze percentage according to said test subject's eye fixation time; and,
determining a gaze score for each combination of gender and age group.

24. The method of claim 23 including:
determining the test subjects saccade duration between each displayed image; and,
determining a gaze score according to said fixation duration and said saccade duration.

25. The method of claim 23 wherein providing a set of display sets includes providing a set of display sets where each display set includes an image of an adult male, an adult female, teen male, teen female, prepubertal male, prepubertal female, preschool male and preschool female.

26. The method of claim 23 wherein providing a set of display sets includes providing a set of display sets where each within a display set contains eight images.

27. The method of claim 23 wherein providing a set of display sets includes providing a set of display sets where each within a display set contains images of individuals having a the same ethnic origins.

28. The method of claim 23 wherein providing a set of display sets includes providing a set of display sets where each within a display set contains images of individuals wearing the same attire.

29. The method of claim 23 including:
determining the number of fixations for each image using said eye-tracking sensor; and,
said step of determining a gaze score includes the step of determining a gaze score according to said fixation duration, said gaze duration and said number of fixations.
